(19) United States
(12) Patent Application Publication
(10) Pub. No.: US 2006/0247601 A1
(43) Pub. Date: Nov. 2, 2006

(54) Method of improved drug delivery and for treatment of cellulitis

(76) Inventors: Philip James Ellin, Thrybergh (GB); Darren Charles Swinnerton, Newton (GB)

Correspondence Address: KEELING PATENTS AND TRADEMARKS, 3310 KATY FREEWAY, SUITE 100, HOUSTON, TX, 77007, US

(21) Appl. No.: 11/405,855

(22) Filed: Apr. 18, 2006

Related U.S. Application Data
(60) Provisional application No. 60/672,723, filed on Apr. 19, 2005.

(51) Int. Cl. A61M 31/00

(52) U.S. Cl. 604/500; 604/22

(57) ABSTRACT

A method of improving drug delivery to treat a condition comprises the steps of administering the drug to a patient suffering the condition and applying cycloid vibration to the region of the body where the drug is to be delivered. The vibration is applied for a period of 30 minutes three times per day until the condition is diminished. The cycloidal vibration comprises small amplitude, 0.1 and 0.5 mm, low frequency, 15 to 75 HZ, vibration producing motion in three different directions. The method is effective in the treatment of cellulitis where the drug administered is an antibiotic.

### CROSS-REFERENCE TO RELATED APPLICATIONS

This application claims the benefit of U.S. Provisional Patent Application No. 60/672,723 entitled, “Method of Improved Drug Delivery,” filed on Apr. 19, 2005, and U.S. Provisional Patent Application No. 60/672,713 entitled, “Treatment of Cellulitis, both filed in the United States Patent and Trademark Office.

### STATEMENT REGARDING FEDERALLY SPONSORED

Not Applicable.

### RESEARCH OR DEVELOPMENT

Not Applicable.

### BACKGROUND OF THE INVENTION

1. Field of the Invention

This invention relates to a method improving the delivery of drugs to regions of the body, and in particular to an improved method of treatment of cellulitis.

### BACKGROUND

It is an object of the present invention to provide a method of improving drug delivery to certain regions of the body. Oedema accompanies many conditions it is desired to treat by the administration of drugs. However, oedema is the result of stasis, or at best, results in stasis of the fluid comprising the oedema. Furthermore the swelling of oedema tends to restrict blood and lymph vessels, further enhancing the oedema by failure to transport away the fluid causing the oedema. Cause and effect are somewhat irrelevant here, since it is clear that oedema and low blood/lymph flow are two sides of the same coin. But, of course, drugs are frequently needed where the oedema exists, and yet it is here that the blood and lymph that brings the drug to their destination are slowed down, particularly sites where drug delivery is affected by the presence of oedema.

Indeed, any condition which is treated by administration of drugs, whether topically, transdermally, orally, intravenously or intramuscularly, relies on transportation of the drugs to the site where they are needed, which often is not exactly where they are first applied to the body. Therefore, it is an object of the present invention to improve the delivery of drugs to certain regions of the body administered to the body remotely from said regions and transported to such regions by natural processes pertaining in the body. Such processes include transportation by the vascular system, the lymph system or simple diffusion.

Cellulitis, a common skin infection, is one such condition. In 2002 to 2003, in the UK, there were nearly 60,000 recorded admissions into hospital (1). Each admission can take on average 10 days to treat (2), accounting for up to six hundred thousand-bed days per annum.

Most commonly affecting the lower limbs, cellulitis is an acute infection of the skin and subcutaneous tissues, characterised by: local heat, redness, pain, erythematous tissue and swelling (2,3).

It is commonly caused by the bacteria streptococci and is associated with or can be a consequence of lower limb swelling/oedema. This can be due to a mix of any of the following: leg oedema, venous hypertension, lymphoedema, chronic ulceration and immobility (4). Koutkia et al (5) conducted a prospective survey to evaluate factors that might contribute to the development of cellulitis. The authors noted that the majority of patients had predisposing factors including oedema and peripheral vascular disease.

Antibiotics, either intravenously or orally administered, and immobilisation are most commonly used methods to treat cellulitis on admission into hospital. Any blistering or exudate is managed with a non-adherent dressing. Antibiotics will be used to treat the infection, however many patients, due to the mix of their oedema and infection can experience extended periods of hospitalisation (10 days plus) (6), which is a heavy burden on hospital resources.

It is therefore an object of the present invention to provide a method of treatment of cellulitis that improves the current position.

### BRIEF SUMMARY OF THE DISCLOSURE

In accordance with a first aspect of the present invention, there is provided a method of improving drug delivery to treat a condition, said method comprising the steps of:

administering one or more drugs to a patient suffering the condition; and

applying a vibration pad to the region of the body where the drug is to be delivered through the body of the patient from its site of application and submitting the pad to cycloid vibration for a period of at least 30 minutes at least once per day until the condition is diminished.

In accordance with a second aspect of the present invention, there is provided a method of treatment of cellulitis comprising the steps of:

administering one or more antibiotics; and

applying a vibration pad to the region of the skin affected by cellulitis and submitting the pad to cycloid vibration for a period of at least 30 minutes at least once per day until the infection diminishes.

Cycloidal vibration is a small amplitude, low frequency, vibration that produces motion in three different directions. The amplitude of such vibration is typically in the range of 0.1 and 0.5 mm with a frequency typically in the range of 15 to 75 Hertz. Each of the three different directions of motion is created at different points in the cycle. It is the “out of phase” relationship which gives rise to the term cycloid vibration. Devices which generate such cycloid vibration are known. GB-A-2096899 and U.S. Pat. No. 3,019,785, the full disclosures of which are herein incorporated by reference, disclose a vibration pad device comprising a motor mounted in a frame, the frame extending into a pad and the motor driving an eccentric weight that causes cycloid vibration of the pad. Cycloid vibration can be administered by means of integration of the mechanism into static products such as a portable pad as disclosed in GB-A-2096899, but equally it can be incorporated in a mattress, of a therapy couch, for example.

Vibro-Pulse® therapy (provided by Vibrant Medical Limited, Sheffield, England, assignees of the present invention) is a form of non invasive cycloidal vibration that stimulates fluid turnover in tissue and increases microcirculation and blood flow without using a compressive force (7). Using non invasive application of cycloidal vibration in addition to traditional forms of therapy, to treat lower limb cellulitis is surprisingly found to reduce treatment time and improve healing rates.

Without being constrained to any particular theory as to why this occurs, it is believed that the increase in blood flow and microcirculation in the capillaries due to cycloidal vibration increases the delivery and effectiveness of the antibiotics (IV and or oral) resolving the cellulitis more quickly. Furthermore, fluid turnover in the tissue gently stimulates the lymphatics, reducing the limb oedema associated with cellulitis. To date stimulating circulation when an infection is present has not been recommended as there is a fear that the infection may spread and cause other complications.

Lievens et al, (8) has found that, following cycloid vibration, the arteries and veins in mice dilated during the treatment. These dilations will stimulate blood flow, therefore enhancing the delivery of drugs in the blood at the site of the area to be treated. The lymphatics, on the other hand, constricted during cycloid vibration, but vasodilated after it. This mechanism would stimulate greater lymphatic function, therefore reducing fluid/oedema within the tissues. It has been shown that cycloid vibration reduces leg fluid volumes where oedema and lymphoedema are present (9,10).

Ryan and Salter (11) explained that cycloid vibration expedites the dispersal of tissue fluid lying between the more solid and vibrating elements of the tissues. Relatively stiff tissue components transmit vibration well, causing fluid to be pumped into and along the lymphatics, reducing tissue pressure, at the same time blood vessels compress and dilate. When the vibration stops blood vessels expand, vibration having decreased both pressure and total volume of the tissue by decreasing the fluid content of the colloidal gel of the dermis.

Cherry et al (12) determined that the use of cycloidal vibration on the lower limb resulted in oedema reduction in patients with venous hypertension.

Many patients with cellulitis also present with respiratory and cardiac problems. As a consequence, it is considered that compressing the patient's legs with the traditional means of compression bandaging would too quickly move too much fluid off the patient's oedema and into the patient's cardiovascular system, therefore increasing the risk of chronic cardiac failure. Using cycloidal vibration gently stimulates circulation and oedema reduction in the patient's lower limbs without affecting the patient's cardiac or respiratory state.

However the present invention suggests the application of vibration to stimulate gentle circulation while the patient is being actively treated for an infection by medication or antibiotic.

The resulting effect is reduction in oedema in the limb. This reduction in fluid takes pressure off the vascular system, allowing the vessels to expand. This in turn enhances blood flow and the delivery of the antibiotics or other medication to the area of infection is increased. This results in the drugs being more effective and treating the infection quicker.

Consequently, it is anticipated that the invention may be generally applied for any treatment using drug delivery through the body, from its point of application to a remote region of the body, which region is capable of responding to the application of such cycloidal vibrations and is responsible for any delay in the delivery of the drug through said region, will be accelerated in its delivery to the region where it is needed. More specifically the present invention found may be utilized in the treatment of oedema.

This applies whether the drug is administered orally, intravenously, subcutaneously, intramuscularly, or topically. Indeed, whenever the point of application of the drug is remote from the point at which it is needed, and whether the route between the two is primarily via the vascular system, the lymph system or simply by diffusion, each transport mechanism seems improved by the application of cycloidal vibration, at least in the region affected by the vibration. Again, whether this is caused by reduction in oedema, in such cases where that exists, which it is known that cycloidal vibration reduces, or accompanies the reduction in oedema is irrelevant. Indeed, the vibration therapy is effective when there is no oedema.

### BRIEF DESCRIPTION OF DRAWINGS

FIG. 1 is a flow diagram showing the steps of an exemplary embodiment of the invention.

### DESCRIPTION OF THE INVENTION

By virtue of the other conditions often accompanying, or pre-existing, cellulitis, it is the leg region of the patient most frequently affected. In this event, the pad is placed on the lower leg.

There is provided a method of improving drug delivery to treat the condition, including administering one or more drugs to a patient suffering the condition and applying a vibration pad to the region of the body where the drug is to be delivered through the body of the patient from its site of application and submitting the pad to cycloid vibration

Cycloidal vibration, a small amplitude, low frequency, vibration that produces motion in three different directions, is applied. The amplitude of such vibration is typically in the range of 0.1 and 0.5 mm with a frequency typically in the range of 15 to 75 Hertz. Each of the three different directions of motion is created at different points in the cycle. Cycloid vibration can be administered by means of integration of the mechanism into static products such as a portable pad as disclosed in GB-A-2096899, but equally it can be incorporated in a mattress, of a therapy couch, for example. A suitable vibration device is also disclosed in WO-A-2002/065973, the full disclosure of which is herein incorporated by reference, in which a strap is employed to tie the pad closely to the patient's leg, so that vibrations are more deeply transmitted to the leg.

Preferably, said application of vibration is conducted three times per day. Preferably, each said application of vibration is at least 30 minutes. Preferably, application of cycloid vibration is effected in the period one-half to two hours after administration of the drug.

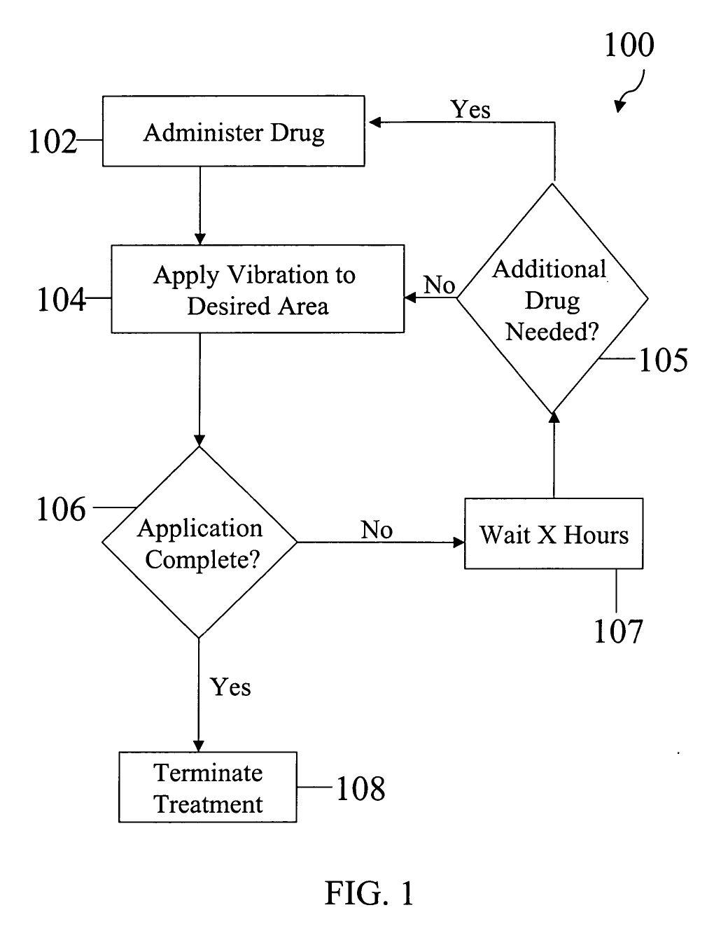

The foregoing is represented by flowchart in FIG. 1. The steps of the preferred invention include step 102, wherein a drug such as antibiotics are administered.

Thereafter, step 104 applies a vibration pad to the region affected by cellulitis. Decision 106 determines whether the application is complete. If the application is not completed, a rest period, step 107, occurs. In the exemplary embodiment, at least 2 hours rest is permitted between each treatment. Decision 108 involves a determination whether to continue drug administration 102 and vibration 104 or just vibration 104. As indicated, the treatment may be continued until a determination is made to terminate treatment, step 109.

Method 100 contemplates that different drugs may be administered at each instance of step 102. Drugs may be administered differently at each instance of step 102 such as administering antibiotics are intravenous administration in a first instance of step 102 and oral administration in a subsequent instance of step 102.

The invention is further described hereinafter, by way of example, with reference to the following non-limiting examples.

### COMPARATIVE EXAMPLES

All patients diagnosed as having cellulitis.

### Example 1

Patient 1 (Female, 80 years old)
Admission—Cellulitis
History—Slight hypertension
Cellulitis—right leg, with swelling/oedema

Treatment—intravenous antibiotics (Benzlpenicillin) for 48 hours, followed by oral antibiotics (Flucoxacillin) according to existing prescribed dosages

| | Day 1 | Day 7 |
|---|---|---|
| Calf Circumference | 34 cm | 32.5 cm |
| Thigh Circumference | 43 cm | 42.5 cm |
| % of erythema/cellulitis reduction | | 50% erythema & swelling |
| | | still present at day 12. |

Patient 2 (Male, 74 years old)
Admission—Cellulitis
History—Ischaemic heart disease
Cellulitis—right leg, swelling

Treatment—intravenous antibiotics (Benzlpenicillin) for 48 hours, followed by oral antibiotics (Flucoxacillin) according to existing prescribed dosages plus Cycloidal Vibration 3× daily for 30 minutes

| | Day 1 | Day 4 |
|---|---|---|
| Calf Circumference | 43 cm | 41 cm |
| Thigh Circumference | 66 cm | 60 cm |
| % of erythema/cellulitis reduction | | 100% |

Summary: Patient 2 had 100% infection reduction by Day 4 plus significant oedema reduction.

### Example 2

Patient 3 (Female, 80 years old)
Admission—Cellulitis, chest infection
History—Ischaemic heart disease
Cellulitis—left leg, with swelling/oedema

Treatment—intravenous antibiotics (Benzlpenicillin) for 48 hours, followed by oral antibiotics (Flucoxacillin) according to existing prescribed dosages

| | Day 1 | Day 7 |
|---|---|---|
| Calf Circumference | 35 cm | 33 cm |
| Thigh Circumference | 52 cm | 52 cm |
| % of erythema/cellulitis reduction | | 50% erythema & swelling |
| | | still present at day 9. |

No swelling reduction after 7 days only 50% cellulitis reduction.

Patient 4 (Male, 74 years old)
Admission—Hip replacement 2 months previously. Constant swelling led to cellulitis
History—Stroke, angina, Ischaemic heart disease, hypertension
Cellulitis—left leg, swelling and blistering

Treatment—oral antibiotics (Fluloxacillin) plus Cycloidal Vibration 3× daily for 30 minutes

| | Day 1 | Day 6 |
|---|---|---|
| Calf Circumference | 37 cm | 32 cm |
| Thigh Circumference | 45 cm | 40 cm |
| % of erythema/cellulitis reduction | | 100% plus significant |
| | | oedema reduction |

Summary: Patient 3, treated with both intravenous and oral antibiotics, still had cellulitis at Day 9, whereas patient 4, treated only with oral antibiotics, as well as cycloid vibration, had 100% cellulitis reduction at Day 6.

### Example 3

Patient 5 (Male, 70 years old)
Admission—Chest infection
History—Anaemia (not long standing, now resolved)
Cellulitis—left leg, swelling/oedema

Treatment—IV antibiotics for 48 hours, then oral antibiotics

| | Day 1 | Day 7 |
|---|---|---|
| Calf Circumference | 31 cm | 29.5 cm |
| Thigh Circumference | 41 cm | 42 cm |
| % of erythema/cellulitis reduction | | 75% plus little oedema |
| | | reduction |

Patient 6 (Female, 88 years old)
Admission—Cellulitis
History—Ischaemic heart disease, hypertension
Cellulitis—severe right leg, swelling

Treatment—oral antibiotics (Metronidazole) plus Cycloidal Vibration 3× daily for 30 minutes

| | Day 1 | Day 7 |
|---|---|---|
| Calf Circumference | 35 m | 32 cm |
| Thigh Circumference | 45 cm | 38.5 |
| % of erythema/cellulitis reduction | | 75%, plus significant |
| | | oedema reduction |

Summary: Patient 5 was treated with both intravenous and oral antibiotics and had 75% reduction in 7 days. Patient 6 had severe cellulitis and was treated only with oral antibiotics plus cycloid vibration treatment, but achieved same level of reduction.

### Example 4

Patient 7 (Male, 44 years old)
Admission—Cellulitis
History—
Cellulitis—left leg, slight swelling/oedema

Treatment—IV antibiotics (Clarithromycin) for 48 hours, then oral antibiotics (Flucloxacillin)

| | Day 1 | Day 7 |
|---|---|---|
| Calf Circumference | 39 cm | 39 cm |
| Thigh Circumference | 52 cm | 54 cm |
| % of erythema/cellulitis reduction | | 50% no oedema reduction, |
| | | and foot/ankle area |
| | | remained red and swollen |

Patient 8 (Male, 63 years old)
Admission—Cellulitis
History—Angina, Ischaemic heart disease
Cellulitis—right leg, swelling

Treatment—intravenous antibiotics (Benzlpenicillin) for 48 hours, followed by oral antibiotics (Flucoxacillin) according to existing prescribed dosages plus Cycloidal Vibration 3× daily for 30 minutes

| | Day 1 | Day 7 |
|---|---|---|
| Calf Circumference | 46 m | 38 cm |
| Thigh Circumference | 52 cm | 45 cm |
| % of erythema/cellulitis reduction | | 75%, and significant |
| | | oedema reduction by Day 6 |

Summary: Patient 8 has suffered with significant severe recurrent cellulitis. Tissue viability nursing staff stated that, hitherto, it had taken up to two months of treatment of this patient to achieve the same results seen in seven days using cycloid vibration treatment.

### FURTHER EXAMPLES

A further series of trials were conducted. In the table below, the results from a randomised controlled trial for the treatment of lower limb cellulitis (infection of the subcutaneous tissue of the skin) comparing standard treatment of Intravenous or Oral Antibiotics to standard treatment plus cycloidal vibration 3 times a day for 30 minutes per treatment. Outcomes to determine amount of cellulitis reduction after 7 days.

Results from the Control Group/Standard Drug Therapy Treatment.

| | | DAY1 | Treatment: | DAY 7 | DAY 7 | Average % limb |
|---|---|---|---|---|---|---|
| | Patient | Ankle/Calf/Thigh | Intravenous (IV) or | % Cellulitis | Ankle/Calf/Thigh | circumference |
| | (sex/age in years) | circumference (cm) | Oral antibiotics | reduction | circumference (cm) | reduction |
| 1 | AG(F/80) | NA/34/43 | IV | 50 | NA/42.5/32.5 | −3 |
| 2 | DB(M/44) | NA/39/52 | IV | 50 | NA/39/54 | +2 |
| 3 | RK(M/70) | NA/31/41 | IV | 75 | NA/29.5/42 | −1 |
| 4 | EG(F/80) | NA/35/52 | IV | 50 | NA/33/52 | −2 |
| 5 | VA(F/75) | 20/25/38 | IV | 0 | 18/25/38 | −2 |
| 6 | DM(F/62) | 25.5/35/38 | IV | 50 | 23/32/38 | −6 |
| 7 | MB(M/46) | 26.5/38/51 | IV | 50 | 26.5/37.5/51 | 0 |
| 8 | RGS(F/65) | 34/40/53 | IV | 75 | 30/39.5/50 | −6 |
| 9 | MM(F/67) | NA/35/42 | Oral | 100 DAY 6 | NA/31/41 | −7 |
| 10 | LP(F/43) | 34/64/86 | IV | 75 | 34/61/87 | −1 |
| 11 | CH(M/42) | 34/61/76.5 | IV | 25 | 38/62/78 | +3 |
| 12 | MB(F/74) | 22/37/47 | IV | 40 | 25/39.5/50 | +4 |
| 13 | IB (F/86) | 26/37/49 | IV | 100 DAY 6 | 22/33/52 | −5% |
| 14 | VK(F/72) | 28/52/60 | IV | 0 | 27.5/50/61 | −1% |
| 15 | ET(M/73) | 27/41.5/61 | Oral | 25 | 28/43.5/58.5 | 0% |
| 16 | MT(F/71) | 25/38/49.5 | Oral | 75 | 25/27.5/49 | −10% |

NA = not applicable (ie not measured)

Results From the Experimental Group of Standard Drug Therapy Treatment Plus Cycloidal Vibration Three Times Daily.

| | | | | DAY 7 | | |
|---|---|---|---|---|---|---|
| | | DAY 1 | Treatment: | (or sooner) | Ankle/Calf/Thigh | Average % limb |
| | Patient | Ankle/Calf/Thigh | Intravenous (IV) or | % Cellulitis | circumference (cm) | circumference |
| | (sex/age in years) | Circumference (cm) | Oral antibiotics | reduction | (at end of treatment) | reduction |
| 1 | KA(M/74) | NA/43/66 | IV | 100 DAY 4 | NA/41/60 | −7 |
| 2 | JC(F/88) | NA/35/45 | Oral | 75 | NA/32/38.5 | −12 |
| 3 | HJM(M/74) | 24/37/45 | Oral | 100 DAY 6 | 19.5/32/40 | −14 |
| 4 | DWP(M/50) | 19.5/33/44 | IV | 100 DAY 2 | 19/31/44 | −3 |
| 5 | PB(M/64) | 22/35/50 | IV | 100 DAY 7 | 21/34/43 | −9 |
| 6 | IH(M/63) | NA/46/52 | IV | 75 | NA/38/45 | −16 |
| 7 | JP(F/80) | 28.5/43/58 | IV | 75 | 27/42/57 | −3 |
| 8 | MP(F/84) | 24/36/53 | IV | 100 DAY 6 | 22/36/54 | −1 |
| 9 | RM(F/82) | 29/36/50 | IV | 100 DAY 6 | 22.5/34/47 | −10 |
| 1 | BB M/75) | 26/41/51 | IV | 100 DAY 5 | 25/38/49 | −5 |
| 11 | AS(M/64) | 25/40/49 | Oral | 50 | 25/39/48 | −2 |
| 12 | GET(M/50) | 32/54/65 | Oral | 25 | 31/53/64 | −2 |
| 13 | MN(M/58) | 27/36/45 | IV | 100 DAY 7 | 27/34/42 | −5 |
| 14 | RR(M/58) | 28.5/41/50 | IV | 100 DAY 5 | 26/40/50 | −3 |
| 15 | MR(F/67) | 27/32/46 | IV | 100 DAY 7 | 23.5/34/45 | −3 |
| 16 | JPM(M/38) | 29/49/74 | Oral | 100 Day 7 | 26/45/66 | −10 |
| 17 | GW(M/70) | 24/40/48 | IV | 100 Day 4 | 24/36.5/45 | −6 |
| 18 | MW(F73) | 25/51/55 | IV | 100 Day 5 | 22/43/53 | −10 |

NA = not applicable (ie not measured)

### Summary of Results

Patients with 100% cellulitis reduction: In the Control group (having standard treatment only) 12% of the patients had 100% cellulitis reduction in an average treatment time of 6.9 days. However, in the Experimental group (having standard treatment plus cycloidal vibration) 72% of the patients had 100% cellulitis reduction in an average treatment time of 5.5 days.
Oedema: In the Control group, the average limb circumference reduction was 2% in 6.9 days. In the Experimental Group, the average limb circumference reduction was 6.8% in 5.8 days.
Erythema/Cellulitis Reduction: In the Control group average cellulitis/erythema reduction was 52% in an average of 6.9 days. This compares to the Experimental group where an average 89% of the cellulitis/erythema reduced in an average of 5.8 days.
   Thus, in the Experimental group 72% of the patients had complete cellulitis reduction in an average period of 5.5 days treatment (compared with 12% after 6.9 days of the Control Group), and the remainder of the Experimental Group had an average cellulitis/erythema reduction of 60% by day 7 (compared 45% for the Control Group).
Conclusion: By stimulating the microcirculation and lymphatics using cycloidal vibration enhanced antibiotic delivery and oedema reduction occurred, reducing the treatment time of cellulitis by up to 50%.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

### REFERENCES

1 English Hospitals Episodes Statistics (Financial year 2002-2003). Department of Health.
2. Cox N, Colver G B and Paterson W D. Management and morbidity of cellulitis of the leg. Journal of the Royal Society of Medicine. 1998, Vol 91, Issue 12 634-637
3. Baxter H, McGregor F. Understanding and Managing cellulitis. Nursing Standard Jul. 18-24, 2001; 15(44):50-2,54-6
4 Morton N, Swartz, M.D. Cellulitis. New England Journal of Medicine 2004; 350:904-12.
5 Dupuy A, Benchikhi H, Roujeau J C, Bernard P, Vaillant L, Chosidow O, Sassolas B, Guillaume J C, Grob J J, Bastuji-Garin S. Risk factors for erysipelas of the leg (cellulitis): case-control study. BMJ. Jun. 12, 1999; 318(7198):1591-4.
6 Koutkia P, Mylonakis E, Boyce J. Cellulitis: Evaluation of Possible Predisposing Factors in Hospitalised Patients. Diagnostic Microbiological Infectious Disease. 1999; 34:325-327
7 Cox N H. Management of lower leg cellulitis. Clinical Medicine. 2002. Vol 2; 23-27
8 Ryan, T, J. Thoolen, M. Yang, Y. The effect of mechanical forces (vibration and external compression) on the dermal content of the upper dermis and epidermis assessed by high frequency ultrasound. J Tiss Viab 2001; 11:3, 97-101
9 Lieven, P. Leduc, A. Dewald, J. The use of multidirectional vibrations on wound healing and on the regeneration of blood and lymph vessels. In: Proceedings of the VII International congress of Lymphology. Prague: Czechoslovak Medical Press, 1981.
10 Wilson, J, M. Arseculeratne, Y, M. Yang, Y. Cherry, G. W. Healing venous ulcers with cycloidal multidirectional vibration therapy. Journal of Wound Care 2002: 11:9 Merritt J, Piller N, Carati C, Bridger B. Home Based Massage Pad—Results of a large scale clinical trial of cyclo massage for chronic primary and secondary leg lymphoedemas. Proceedings of the World Federation of Occupational Therapists. Stockholm June 2002.
11 Ryan T J, Salter. D. The effect of vibration on skin blood flow. Bibliotheca Anatomica 1977; 16: 180-184.
12 Cherry G. W, Wilson J. M, Arseculeratne Y. M, Yang Y. Healing venous ulcers with cycloidal multidirectional vibration therapy. Oxford Wound Healing Institute. Churchill Hospital. Oxford UK. Journal of Wound Care Vol 11, No 10, November 2002

1. A method of treatment of cellulitis comprising the steps of:
administering antibiotics; and
applying a vibration pad to the region of the skin affected by cellulitis and submitting the pad to cycloid vibration for a period of at least thirty minutes at least once per day until the infection diminishes.

2. A method according to claim 1, wherein said cycloidal vibration comprises small amplitudes, 0.1 and 0.5 mm, low frequency, 15 to 75 HZ, vibration producing motion in three different directions.

3. A method according to claim 1, wherein said application of vibration is conducted three times per day.

4. A method according to claim 3, wherein at least two hours rest is permitted between each said application.

5. A method of treatment according to claim 1, wherein said application of cycloid vibration is effected in the period one-half to two hours after administration of the antibiotics.

6. A method of treatment according to claim 1, wherein said patient's leg is treated with said vibration and a strap is employed to tie the pad closely to said patient's leg, so that vibrations are more deeply transmitted to the leg.

7. A method of treatment according to claim 1, wherein said antibiotics are administered intravenously, followed by oral administration.

8. A method of improving drug delivery to treat a condition, said method comprising the steps of:
administering the drug to a patient suffering the condition; and
applying a vibration pad to the region of the body where the drug is to be delivered through the body of the patient from its site of application and submitting the pad to cycloid vibration for a period of at least thirty minutes at least once per day until the condition is diminished.

9. A method according to claim 8, wherein said cycloidal vibration comprises small amplitude, 0.1 and 0.5 mm, low frequency, 15 to 75 HZ, vibration producing motion in three different directions.

10. A method according to claim 8, wherein said application of vibration is conducted three times per day.

11. A method according to claim 10, wherein at least two hours rest is permitted between each said application.

12. A method of treatment according to claim 8, wherein said patient's leg is treated with said vibration and a strap is employed to tie the pad closely to said patient's leg, so that vibrations are more deeply transmitted to the leg.

13. A method of treatment according to claim 8, wherein said application of cycloid vibration is effected in the period one-half to two hours after administration of the antibiotics.
